(19) United States
(12) Patent Application Publication
(10) Pub. No.: US 2025/0387244 A1
(43) Pub. Date: Dec. 25, 2025

(54) COVERED STENT

(71) Applicant: BCM Co., LTD., Gyeonggi-do (KR)

(72) Inventor: Byung Cheol MYUNG, Gyeonggi-do (KR)

(21) Appl. No.: 19/246,696

(22) Filed: Jun. 24, 2025

(30) Foreign Application Priority Data
Jun. 25, 2024 (KR) 10-2024-0082444

(51) Int. Cl. A61F 2/90

(52) U.S. Cl. CPC A61F 2/90; A61F 2210/0014; A61F 2230/0069

(57) ABSTRACT

Disclosure relates to a covered stent preventing an opening of an extension tube from being collapsed when used in a lesion area, facilitating forward movement of an outer tube, and allowing the outer tube to easily pass through a narrow lesion area. The covered stent includes a cylindrical stent made of shape memory alloy wires woven into a hollow cylindrical mesh shape to form multiple space parts within the wires, a connecting part extending from one or both ends of the stent and bent externally, an extension tube extending from one end of the connecting part and having a cylindrical shape with a greater diameter than the stent, a first film formed by bonding first and second polytetrafluoroethylene tapes formed at the stent without one end of the extension tube, the connecting part, and the extension tube, and a silicone second film formed one end of the extension tube.

### CROSS REFERENCE TO RELATED APPLICATION

The present application claims priority to Korean Patent Application No. 10-2024-0082444, filed Jun. 25, 2024, the entire contents of which are incorporated herein for all purposes by this reference.

### BACKGROUND OF THE INVENTION

### Field of the Invention

The present disclosure relates to a covered stent used to expand a passage with a stenotic or occlusive lesion area occurring in a lumen of the human body.

### Description of the Related Art

In general, when a stenotic or occlusive lesion occurs in a lumen of the body, such as the esophagus, duodenum, biliary tract of the human body, and the like, the inherent function of the lumen to move fluid is decreased.

Therefore, a stent is inserted into a portion with a stenotic or occlusive lesion to expand the narrowed lumen. In this respect, Patent document 1 provides a stent formed by a method for forming a polytetrafluoroethylene (PTFE) film on the stent, including: taping a first PTFE tape around entire outer surfaces of a stent having multiple space portions and a jig formed with the same shape as the stent; after inserting the jig into the stent, wrapping a second PTFE tape around the entire outer surfaces of the stent; inserting the stent with the jig inserted therein into an oven and heating the stent so that the first and second PTFE tapes are bonded together, and then removing the stent from the oven; inserting heated jig and stent wrapped with the 1 and 2 PTFE tapes into a first insertion part of a lower elastic member having a same shape as a portion of the jig, and inserting it into a first insertion part of an upper elastic member having a same shape as a remaining portion of the jig; inserting the lower elastic member and the upper elastic member into the first insertion parts of molds having the same shape as the lower elastic member and the upper elastic member, and then compressing the upper elastic member with a pressure member to allow the lower elastic member and the upper elastic member to receive pressure in all directions from the molds and the pressure member, and bonding the first and second PTFE tapes together to form a PTFE film on the stent and fill the multiple space portions of the stent; and releasing pressure of the pressure member and removing the lower elastic member and the upper elastic member from the first insertion parts of the molds, and after removing the stent from the first insertion part of the lower elastic member and the first insertion part of the upper elastic member and then removing the jig from the stent.

However, in Patent document 1, the PTFE film is weakly elastic, so there is a concern that the resilience of the stent would be decreased.

For this reason, as shown in FIGS. 1A and 1B, when the stent on which the PTFE film is formed is applied to a stenotic or occlusive lesion occurring in a lumen of the body, there is a concern that an opening of an extension tube part formed at either portion of the stent would be collapsed without completely unfolding due to the stent with the resilience decreased by the PTFE film.

Furthermore, there is a concern that fluids moving along the lumen of the body would get stuck in the obstructed opening of the extension tube part and not be able to move smoothly along the lumen 1 of the body.

Therefore, conventionally, as shown in FIG. 2, instead of the PTFE film, a silicone film having great elasticity is formed on the entire portion of the extension tube part of the stent, thereby increasing the resilience of the stent to solve the problem of the extension tube part with the collapsed opening.

However, as shown in FIG. 3, since the silicone film is formed on the entire portion of the extension tube part, the thickness of the extension tube part with the silicone film is greater than the thickness of the existing extension tube part with the PTFE film.

For this reason, as shown in FIG. 4, when the stent is inserted into a mounting space of a delivery system and then the external tube moves forward, the external tube is brought into wide contact with the silicone film of the extension tube part to create a large amount of resistance. This makes it difficult for the external tube to move forward, making it difficult for the stent to be mounted to the mounting space of the delivery system.

Therefore, with an external tube having a greater diameter than the diameter of an external tube, it is possible to mount the stent to the mounting space of the delivery system, but as shown in FIG. 5, it is difficult to make the passage of the stent as an increased diameter of the external tube 2a is obstructed by the narrowed lesion 1a occurring in the lumen 1 of the body to increase a resistance.

### Document of Related Art

### Patent Document

Patent Document 1: Korean Patent No. 10-2535715 (May 18, 2023)

### SUMMARY OF THE INVENTION

Accordingly, an objective of the present disclosure is to provide a covered stent different from a conventional stent, the covered stent being capable of allowing an opening of an extension tube part to be fully opened without being collapsed when used in a lesion area, facilitating forward movement of outer tube so as to be easily mounted to a mounting space of a stent delivery system, and allowing the outer tube to easily pass through a narrow lesion area while excluding an outer tube having a large diameter when using the stent.

In order to achieve the above-described objective, according to the present disclosure, there is provided a covered stent, the covered stent including: a cylindrical stent formed such that shape memory alloy wires are woven or intersected together into a hollow cylindrical mesh shape with multiple space parts formed within the wires; a connecting part extending from one end or both ends of the cylindrical stent and bent externally; an extension tube part extending from one end of the connecting part into a cylindrical shape so as to have a greater diameter than the cylindrical stent; a first film formed by bonding first and second polytetrafluoroethylene (PTFE) tapes together, the tapes being formed on inner surfaces and outer surfaces of the cylindrical stent without one end of the extension tube part, the connecting part, and the extension tube part; and a silicone second film formed one end of the extension tube part.

According to the present disclosure, the PTFE first film is formed at the cylindrical stent, the connecting part, and the extension tube part, but instead of the PTFE first film, the silicone film is formed at one end of the extension tube part. Therefore, when the stent is used on a lesion area, the opening of the extension tube part can be fully unfolded due to the elasticity of the silicone second film, without being collapsed unlike the conventional stent.

For this reason, the covered stent can easily extend to widen the passage at the lesion area, and the extension tube part with the opening fully unfolded can be correctly obstructed by the lumen in the body and held.

Furthermore, body fluid can flow smoothly along the lumen in the body without being obstructed by the opening of the extension tube part.

According to the present disclosure, since the silicone second film formed at one end of the extension tube part has a length ranging from 1 to 2 mm shorter than the conventional stent, the outer tube is brought into contact with the second film of the extension tube part within a small area, that is, into less contact therewith, thereby reducing the resistance to facilitate forward movement.

For this reason, the covered stent can be easily mounted to the mounting space of the stent delivery system.

According to the present disclosure, an outer tube having a large diameter is not used, so an outer tube having a small diameter can easily move through a narrow lesion area.

For this reason, the use of the covered stent can be easily performed.

According to the present disclosure, resistance is reduced as the outer tube is brought into contact with the second film of the extension tube part within a small area, that is, into less contact therewith, so that rearward movement can be easily performed. Therefore, the covered stent can be easily removed from the mounting space in the stent delivery system.

For this reason, the covered stent can easily expand and widen a passage with a lesion.

### BRIEF DESCRIPTION OF THE DRAWINGS

FIGS. 1A to 5 are operational condition views of a conventional stent,

FIGS. 6 to 9 are a front view and a partial enlarged view of the covered stent according to the embodiment of the present disclosure, and

FIGS. 10 to 14 are operational condition views of the covered stent according to the embodiment of the present disclosure.

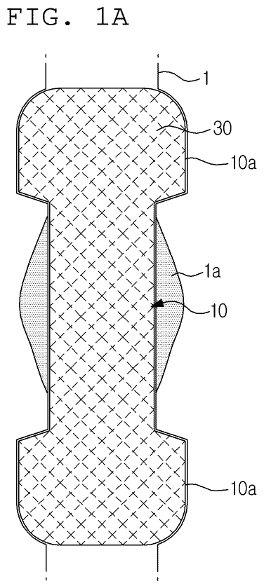

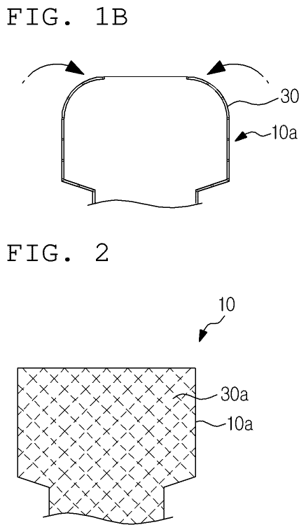

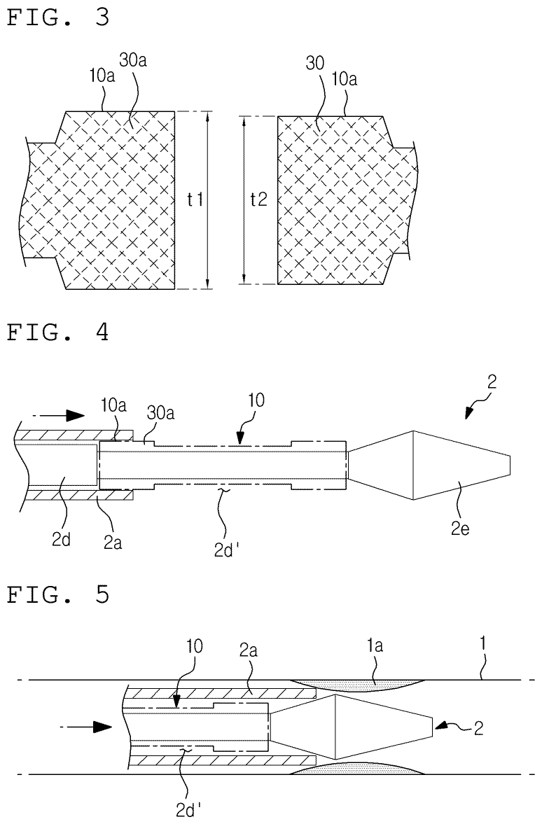

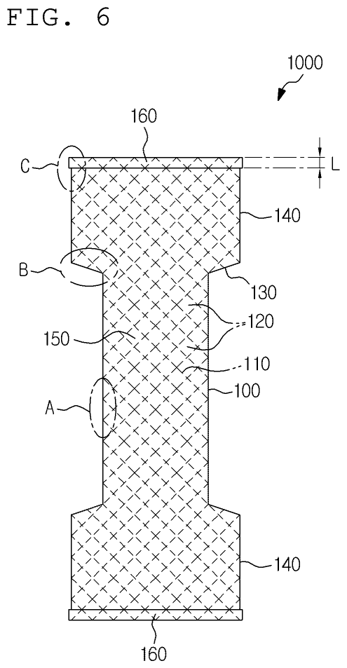

### DETAILED DESCRIPTION OF THE DISCLOSURE

Accordingly, exemplary embodiments of the present disclosure will be described in detail with reference to the accompanying drawings as follows.

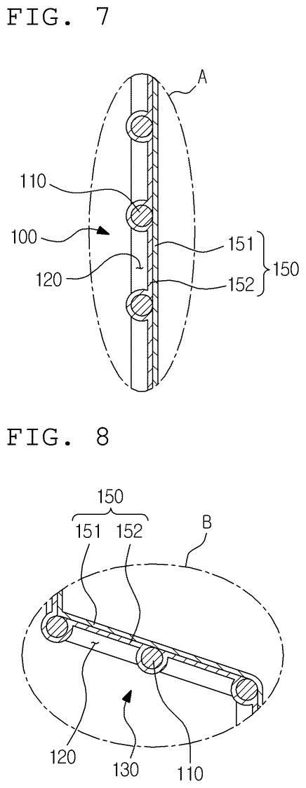

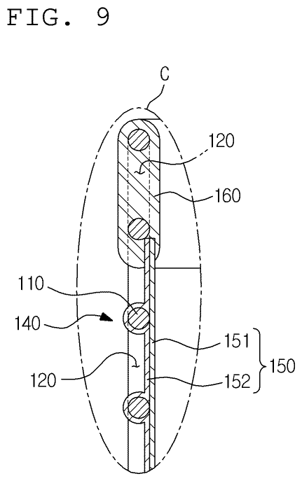

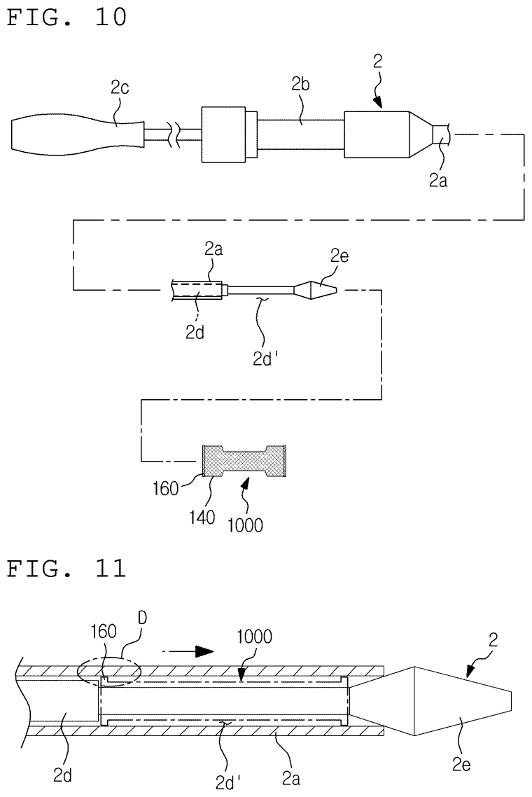

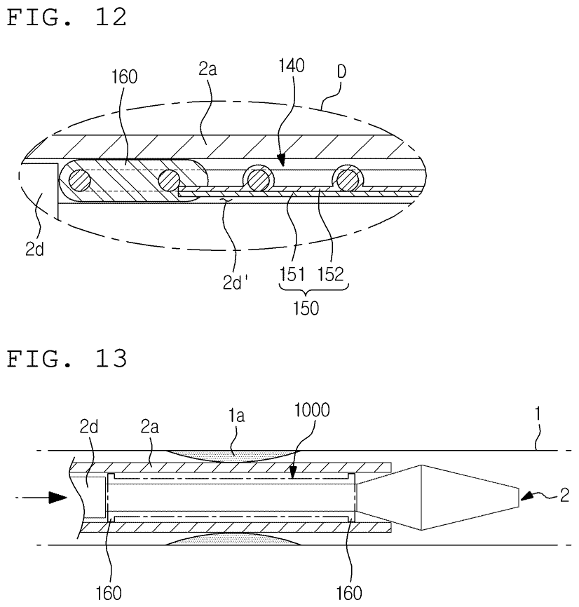

As shown in FIGS. 6 to 14, according to the embodiment of the present disclosure, a covered stent 1000 is inserted into a lumen 1 of the human body, such as the esophagus, duodenum, biliary tract of the human body, and the like, with a stent delivery system 2, and is used to expand a stenotic or occlusive lesion area 1a occurring in the lumen 1.

At this point, the stent delivery system 2 includes: a first handle 2b connected to an external tube 2a that moves along the lumen 1 in the human body; a second handle 2c moving behind the first handle 2b; an internal tube 2d connected to the second handle 2c and movably inserted into the external tube 2a, and having a mounting space 2d′, in which the covered stent 1000 of the present disclosure is pressed to the external tube 2a and mounted, at one portion thereof; and a guide tip 2e connected to the internal tube 2d and exposed from the external tube 2a.

The covered stent 1000 includes: a cylindrical stent 100 formed such that wires 110 made of a shape memory alloy are woven or intersected together into a hollow cylindrical mesh form with multiple space parts 120 formed within the wires 110; and a connecting part 130 extending from one end or both ends of the cylindrical stent 100 and bent externally.

At this point, as shown in the drawings, the connecting part 130 is formed at each end of the cylindrical stent 100, but may be formed only at one end of the cylindrical stent 100.

The covered stent 1000 includes: an extension tube part 140 extending one end of the connecting part 130 into a cylindrical shape so as to have a diameter greater than the diameter of the cylindrical stent 100; and a first film 150 formed by bonding first and second polytetrafluoroethylene (PTFE) tapes 151 and 152 together, which are formed on inner surfaces and outer surfaces of the cylindrical stent 100 without one end of the extension tube part 140, the connecting part 130, and the extension tube part 140.

At this point, the first PTFE tape 151 is wrapped around an outer surface of a jig, and the jig is inserted into the covered stent 1000 and then the second PTFE tape 152 is wrapped around an outer surface of the covered stent 1000, and the covered stent 1000 with the jig inserted therein is heated and pressed until the first and second PTFE tapes 151 and 152 be bonded together, and then the first film 150 is formed on the cylindrical stent 100 without one end of the extension tube part 140, the connecting part 130, and the extension tube part 140.

The covered stent 1000 may include a silicone second film 160 formed on one end of the extension tube part 140.

At this point, the second film 160 is formed on one end of each extension tube part 140, i.e., both ends of the covered stent 1000 by being immersed in or spray-coated with a silicon solution.

The longitudinal length of the second film 160 ranges from 1 to 2 mm.

At this point, in describing a reason of forming the length L of the second film 160 within the range from 1 to 2 mm in detail, when the covered stent 1000 with the length L of the second film 160 formed 2 mm or more is inserted into the mounting space 2d′ of the stent delivery system 2 and then the external tube 2a moves forward, the external tube 2a is brought into wide-area contact with the silicone second film 160 formed 2 mm or more on the extension tube part 140, which may create a large amount of resistance. Accordingly, the covered stent 1000 may be mounted to the mounting space 2d′ of the stent delivery system 2 with difficulty.

In other words, in order to mount the covered stent 1000 to the mounting space 2d′ of the stent delivery system 2 easily, the contact area between the external tube 2a and the second film 160 of the extension tube part 140 should be reduced so that the resistance should be reduced to facilitate forward movement thereof, so the length L of the second film 160 ranges from 1 to 2 mm.

Furthermore, the space parts 120 of the covered stent 1000 are closed by the PTFE first film 150 and the silicone second film 160.

According to the embodiment of the present disclosure, the operation of the covered stent 1000 will be described as follows.

As shown in FIGS. 6 to 14, according to the embodiment of the present disclosure, the covered stent 1000 is inserted into the mounting space 2d′ of the stent delivery system 2, and then the external tube 2a moves forward with the first handle 2b.

Thereafter, the external tube 2a moving forward is brought into contact with the silicone second film 160 formed on one end of the extension tube part 140, but since the silicone second film 160 is formed on one end of the extension tube part 140 with the length L ranging from 1 to 2 mm, the external tube 2a is brought into narrow contact with the silicone second film 160 having the length L of 1 to 2 mm on one end of the extension tube part 140, i.e., less contact therewith, so contact resistance is reduced to facilitate forward movement.

For this reason, the covered stent 1000 is compressed by the external tube 2a of the stent delivery system 2 and easily mounted to the mounting space 2d′.

Furthermore, after the location of the lesion area 1a is confirmed by endoscopy, the first, second handle 2b, 2c of the stent delivery system 2 moves forward so that the inner, outer tube 2d, 2a moves forward toward the lesion area 1a along the lumen 1 in the human body.

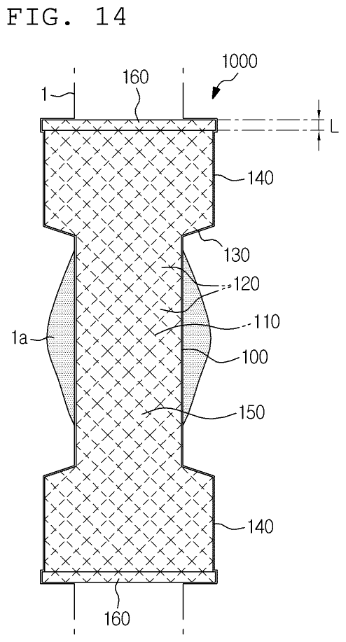

As shown in FIG. 13, the external tube 2a is not replaced with the external tube 2a having a large diameter due to the silicone second film 160 having the length L of the range from 1 to 2 mm on one end of the extension tube part 140, and therefore the external tube 2a is less caught by the narrow lesion area 1a to reduce the resistance to enable the movement to be easy.

Furthermore, the external tube 2a is pulled with the first handle 2b so that the external tube 2a moves rearward.

Thereafter, the external tube 2a moving rearward is brought into contact with the silicone second film 160 formed at one end of the extension tube part 140. However, since the length L of the silicone second film 160 ranges from 1 to 2 mm at one end of the extension tube part 140, a contact area between the external tube 2a and the silicone second film 160 formed at one end of the extension tube part 140 with the length L from 1 to 2 mm is narrow, i.e., the external tube 2a and the silicone second film 160 are brought into less contact with each other. Accordingly, resistance is reduced so that rearward movement of the external tube 2a is easy.

As shown in FIG. 14, the opening of the extension tube part 140 fully unfolds without being collapsed due to the elasticity of the silicone second film 160.

For this reason, the covered stent 1000 of the present disclosure extends easily to widen a portion with the lesion area 1a.

Although the specific embodiments of the present disclosure have been provided, the present disclosure is not limited to the above-described embodiments, and various modifications, equivalents, additions and substitutions are possible by one of ordinary skill in the art to which this present disclosure belongs, without departing from the scope and spirit of the present disclosure.

What is claimed is:

1. A covered stent comprising:
a cylindrical stent (100) formed such that shape memory alloy wires (110) are woven or intersected together into a hollow cylindrical mesh shape with multiple space parts (120) formed within the wires (110);
a connecting part (130) extending from one end or both ends of the cylindrical stent (100) and bent externally;
an extension tube part (140) extending from one end of the connecting part (130) into a cylindrical shape so as to have a greater diameter than the cylindrical stent (100);
a first film (150) formed by bonding first and second polytetrafluoroethylene (PTFE) tapes (151, 152) together, the tapes being formed on inner surfaces and outer surfaces of the cylindrical stent (100) without one end of the extension tube part (140), the connecting part (130), and the extension tube part (140); and
a silicone second film (160) formed one end of the extension tube part (140).

2. The covered stent of claim 1, wherein a longitudinal length (L) of the second film (160) ranges from 1 to 2 mm.
